(19) United States
(12) Patent Application Publication
(10) Pub. No.: US 2018/0258501 A1
(43) Pub. Date: Sep. 13, 2018

(54) Systems And Methods For Identification And Differentiation Of Viral Infection

(71) Applicant: Nantomics, LLC, Culver City, CA (US)

(72) Inventors: Shahrooz Rabizadeh, Los Angeles, CA (US); Kayvan Niazi, Agoura Hills, CA (US); Stephen Charles Benz, Santa Cruz, CA (US); Andrew Nguyen, San Jose, CA (US)

(21) Appl. No.: 15/527,930

(22) PCT Filed: Nov. 20, 2015
(86) PCT No.: PCT/US2015/061970

§ 371 Date: May 18, 2017

Related U.S. Application Data
(60) Provisional application No. 62/083,125, filed on Nov. 21, 2014.

(51) Int. Cl. C12Q 1/70; C12Q 1/68; G06F 19/22; G06F 19/20

(52) U.S. Cl. CPC C12Q 1/70; G06F 19/20; G06F 19/22; C12Q 1/68

(57) ABSTRACT

Systems and methods are provided for determination of primers for differentiation of at least two unspecified pathogens that belong to distinct pathogen (e.g., virus) families that comprise multiple distinct pathogen species and/or varieties.

This application claims the benefit of provisional application having serial number U.S. 62/083,125, filed on Nov. 21, 2014, which is incorporated by reference herein.

### FIELD OF THE INVENTION

The field of the invention is diagnostic systems and methods for rapid identification and differentiation of viral infections, especially as it relates to infection with Ebola virus and differentiation from a symptomatically similar Influenza virus infection.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

In the United States, seasonal influenza A (“flu”) is believed to infect between 5-20% of the population annually resulting in about 200,000 hospital admissions and about 39,000 flu-related deaths. During pandemic periods such as the most recent 2009-2010 “Swine Flu”, these numbers have risen by an additional 43-89 million cases (resulting in between 8,870 to 18,300 additional estimated influenza related deaths). In addition to influenza A, ebolaviruses also demonstrate pandemic potential and share early clinical symptomology with influenza A infection, including the presence of fever, muscle/body aches, headaches, and severe fatigue. Such similarity poses particular challenges where a flu epidemic coincides with presence or suspicion of Ebola infections. Rapid identification and differentiation of the viral pathogen is of utmost importance as clinical care and epidemiological containment of influenza patients (e.g., home care with antiviral drug) differs significantly from protocols required for Ebola patient care (e.g., largely dependent on quarantine with palliative support).

Diagnosis of Ebola infection can be performed in numerous manners and uses viral nucleic acid detection methodologies in most cases. For example, detection of many filovirus species by reverse transcription-polymerase chain reaction using a primer set specific for the viral nucleoprotein gene has been reported to rapidly cover a large variety of virus species that include Ebola and Ebola-related viruses (J Virol Methods. 2011 January; 171(1): 310-3). Such method advantageously allows quick analysis, but unfortunately fails to provide differential diagnostic value against other non-filoviruses. In another approach of viral diagnosis, total RNA from patient serum was subjected to PCR amplification followed by next generation sequencing (Virology 2012 Jan. 5; 422(1):1-5). While powerful and potentially suitable for differentiation of Ebola against influenza, such technology tends to require extensive sample handling and is often associated with substantial cost. In yet another known approach to identify a virus in a sample, PCR amplification is used to produce products that are then aligned against known phylogenetic trees (J Clin Microbiol 2007 January; 45(1):224-6). However, such approach is generally not suitable for identification of viruses with large phylogenetic distance.

Further known PCR protocols for identification of influenza or Ebola viruses typically use multiplex PCR with sets of primers that help identify the presence of various virus strains in a diagnostic panel (see e.g., Clinical Infectious Diseases 1998; 26:1397-1402; J Clin Microbiol 1999 January; 37(1):1-7; J Clin Microbiol 1999 January; 37(5):1352-1355; J Clin Microbiol 2007 February; 45(2):584-589). While such test panels advantageously allow for highly specific differential diagnoses, virus and serotype specific primers needed. Unfortunately, while very specific to a particular virus, the primers may not be specific to other viral variants, especially new variants or variants with high diversity. Moreover, where detection of multiple viruses of the same class is desired, primer complexity rapidly exceeds the human capability of rational-designed primer sets.

To accommodate for such complex task, various software tools have been developed. For example, Greene SCPrimer is a software suite (see e.g., Nucleic Acid Res. 2006, Vol 34, No. 22 p: 6605-6611) that generates in a first step a phylogenetic tree of all sequences for a virus family to identify candidate primers and then runs a greedy set covering problem (SCP) algorithm to so arrive at minimum primer sets that are then further pruned to match melting points for forward and reverse primers. While facilitating selection of primer pairs, such analysis is computational complex and may still not cover all viruses in a viral family or species. Moreover, such method still requires use of degenerate primers, which increases risk of non-specific binding. Still further, such methods also tend to become problematic where viral target sets are very diverse.

To overcome difficulties with diverse target sets while avoiding multiple sequence alignments, a multiplex primer prediction (MPP) tool was developed (see e.g., Nucleic Acid Res. 2009, Vol 37, No. 19, p: 6291-6304) that also uses a greedy algorithm to identify on a family level consensus primers. While conceptually similar to the above approach, the MPP tool does typically not require degenerate primer sequences, but produces relatively short primers (10 nucleotides), which increases the likelihood of non-specific binding and priming of human or human-hosted non-human sequences (e.g., bacterial or viral due to infection).

Thus, while several methods for detection of the Ebola or Influenza virus are known in the art, all or almost all of them suffer from one or more disadvantages. Therefore, there is still a need to provide an improved detection system, especially where differential diagnosis is needed for distinction of a viral infection with Ebola virus or Influenza virus. Such need is further compounded where the diagnosis has to cover multiple strains of the Ebola virus and Influenza virus in a single sample. Therefore, due to the relatively large diversity of viral sequences, there is also an urgent need for systems and methods to quickly identify suitable oligonucleotide sequences with high specificity towards a pathogen and non-hybridization to human DNA under assay conditions.

### SUMMARY OF THE INVENTION

The inventors have discovered that primers for distinct classes of pathogens can be determined using consensus sequences from members of the distinct classes. Most typically, the consensus sequences are obtained by successive alignment and processing of the various pathogen sequences, correction for human sequences, and set difference operation between the respective primers for the distinct classes.

In one aspect of the inventive subject matter, the inventors contemplate a method of obtaining sets of primers for differentiation of two unspecified pathogens. In contemplated methods, each of the unspecified pathogens belong to distinct phylogenetic pathogen (e.g., virus) families, and each pathogen family comprises multiple distinct pathogen species and/or serotypes. Most preferably, contemplated methods include a step of performing respective multiple sequence alignments, via an alignment device, for a plurality of digitally represented genomes of the pathogen species and serotypes of the respective distinct pathogen families to produce an alignment output for each of the distinct pathogen families. Such methods will also include a step of identifying in each alignment output respective consensus sequences having (i) a homology above a minimum threshold, (ii) a length above a minimum threshold, and (iii) a melting temperature above a minimum threshold. In yet another step, identified consensus sequences are collected into respective adjusted alignment outputs for the distinct pathogen families, and in a still further step, sequences are eliminated from the respective adjusted alignment outputs sequences (the eliminated sequences will have a minimum homology to human and human-hosted sequences) to so form respective virus-specific alignment outputs. A set difference analysis is then performed on the virus-specific alignment outputs to so obtain respective sets of consensus sequences for the unspecified pathogens, and primer sequences are then selected from the respective sets of consensus sequences.

Most preferably, the multiple sequence alignment is performed using Clustal X, Clustal W, or Clustal Omega. It is further contemplated that the minimum threshold for the homology is at least 97% (and in some cases the homology is 100%), the minimum threshold for the length is at least 15, and more typically at least 20, and most typically at least 25 bases, while the minimum threshold for the melting temperature is at least 60° C., and more commonly at least 65° C.

In further contemplated aspects, an analysis engine is programmed/configured to process a formatted output from Clustal X, Clustal W, or Clustal Omega, containing alignments of several different members of a pathogen family or order (e.g., influenza A and influenza B). By comparing the alignment of nucleobases of every viral member, regions of conservations can be found. These regions of conservation are then collected for development of primer sequences to uniquely identify all members of a class of pathogens. In cases where regions of conservation are not found or that the regions fail to yield oligomers that meet the melting temperature requirements, this algorithm will allow minimal mismatch bases to reach the desired melting point. Each base position within the alignment is an assigned a conservation score such that upon addition of mismatch bases, the base position that are most representative of viral class will be used to allow the largest degree of compatibility. The primer sequences are then filtered using BlastN to remove any potential mapping to human sequences, reducing the false positive rate. Subsequent analysis the removes sequences that overlap. It is further generally preferred that the step of eliminating is performed using BlastN, wherein the minimum homology to human and human-hosted sequences (e.g., viral sequence known or suspected to be present in the human) is at least 90%, and more typically at least 95%. While not limiting to the inventive subject matter, the set difference analysis is performed using Set Difference and Set Union operations, and/or that the primer sequences are selected to produce an amplicon has a length of between 100 and 800 bases. It is also contemplated that the unspecified pathogens belong to two distinct phylogenetic orders. Finally, it is contemplated that methods may further include a step of determining a primer sequence to produce a cDNA from a viral RNA, and/or that the set of primers comprises between one and five primer pairs for each of the unspecified pathogen.

### BRIEF DESCRIPTION OF THE DRAWING

FIG. 1 is an exemplary schematic flow diagram of a computer founded method of primer identification according to the inventive subject matter.

### DETAILED DESCRIPTION

The inventors have now discovered systems and methods for multiplexed differential detection of one or more strains of a pathogen (e.g., Ebola virus) against one or more strains of another pathogen (e.g., Influenza virus) in a single sample where it is not known a priori which of the pathogen(s) and/or strains are present. Most typically, the detection is based on sets of oligonucleotides targeting various symptomatically similar viruses, and especially Ebola virus (Zaire) and influenza A in biological samples, which are most commonly whole blood or serum, or plasma. In especially preferred aspects of the inventive subject matter, all of the oligonucleotides target highly conserved areas (in some cases 100% conserved across every strain), and have a melting point Tm≥65° C.

It should be particularly noted that in contrast to other multiplex assays contemplated assay are not used to differentiate and identify a single serotype among a choice of many, but to distinguish between unknown (unspecified) classes of pathogens while covering most or all of the serotypes within each class. Moreover, contemplated primers will also be selected such as to exclude non-target specific binding, and especially binding to the host genome or nucleic acids expected or known to be present in the host genome. Lastly, primers are selected such that primer dimers and cross hybridization (i.e., primer designed for first class of pathogen binds to second class of pathogen) is avoided.

As is further shown in more detail below, the inventors used a conceptually simple and effective computer implemented algorithm to identify unique target sequences against which to design the primer panels (in either the complementary or reverse complementary orientation) to so enable rapid identification of influenza A and/or Ebola virus containing samples. Of course, it should be appreciated that while the examples below demonstrate utility for Ebola and influenza virus detection, contemplated systems and methods will be suitable for any pathogens as the inventive subject matter is independent of the specific sequence information. Moreover, it should be noted that the systems and methods described herein are suitable to rapidly identify numerous target sites (and with that individual and multiplex possibilities), enabling an end user to select the best primer pair(s) for their particular amplification platform.

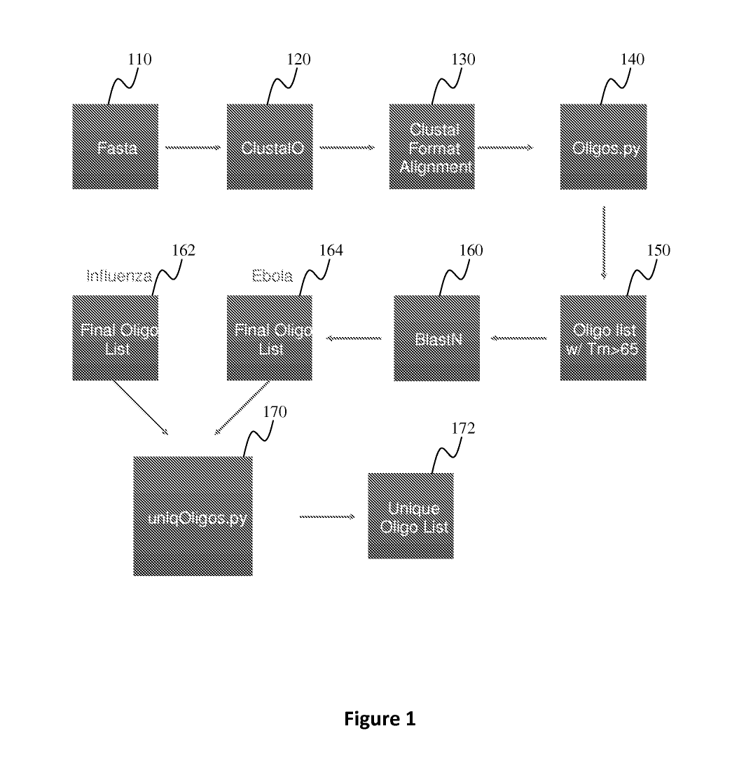

FIG. 1 exemplarily shows a typical computer implemented work flow according to the inventive subject matter. However, it should be noted that all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Here, in a first step 110, all available nucleic acid sequences for a class of pathogens (e.g., all members of a pathogen genus or species serotypes) are obtained from one or more sources, typically from sequence databases in a digital FASTA format. There are many such databases known in the art, and all of the databases are deemed suitable for use herein. Moreover, it should be noted that the data format need not necessarily be limited to FASTA format, and various alternative formats are also contemplated, including FASTQ, BAM, SAM, EMBL, GCG, GenBank, RAW, IG, etc. However, FASTA format is generally preferred and it should be recognized that other formats can be re-formatted to FASTA format where so desired.

Likewise, it should be appreciated that the databases may provide the data online or that the database may be a physical file on CD-ROM, EPROM memory, etc. Regardless of the manner of provision, the sequence data are preferably acquired by an analysis engine that is configured to allow for a rapid alignment of multiple sequences as shown in step 120. Most preferably such alignment is a multi-sequence alignment, and especially preferred aligners include those that use seeded guide trees and HMM profile-profile techniques to generate alignments, such as ClustalO, to produce a corresponding alignment in Clustal format 130. Alternatively, alignments may also be performed in numerous alternative manners and may use alignments based on local regions (e.g., Kalign), based on fast Fourier transforms (e.g., MAFFT), or based on phylogeny-aware methods (e.g., WebPRANK).

Regardless of the particular method, it is therefore contemplated that all sequences for a single class of pathogens are processed to produce a multiple sequence alignment, which is then further processed in step 140 to generate a collection of suitable sequences 150 in which all members satisfy predetermined parameters. Processing step 140 preferably uses the entire multi-sequence alignment in an approach where consensus sequences for the entire alignment are identified that have (i) a homology above a minimum threshold, (ii) a length above a minimum threshold, and (iii) a melting temperature above a minimum threshold.

Most typically, consensus sequences are incrementally identified for areas where the homology is at or above a predetermined threshold (e.g., at least 90%, at least 95%, at least 97%, at least 99%, or 100%) for a predetermined minimum or maximum length (typically between at least 15 bases, at least 20 bases, at least 25 bases, at least 30 bases, at least 40 bases, at least 50 bases, etc., and less than 100 bases, less than 90 bases, less than 70 bases, etc.). As will be readily appreciated, length determination will also be a function of a desired melting point. Most typically, the melting temperature is at least 60° C., or at least 65° C., or at least 68° C. Therefore, in at least some aspects of the inventive subject matter, the consensus sequences so identified will have a minimum threshold for length of at least 20 bases, a minimum threshold for homology of at least 97%, and a minimum threshold for the melting temperature is at least 60° C.

With respect to predetermined homology, it is contemplated that a user will provide the desired minimum threshold, and in most cases the minimum threshold will be above 90% homology. For example, suitable minimum homology thresholds include 95%, 96%, 97%, 98%, 99%, and 100%. As should be readily appreciated, the degree of minimum homology will be a function of the diversity and number of class members for the pathogen, and it is generally preferred that the minimum threshold is lower (e.g., at least 95%) for a more diverse class whereas relatively conserved classes may have a higher minimum threshold (e.g., at least 98%).

Similarly, with respect to the predetermined length, it is contemplated that a user will provide the necessary minimum length and suitable minimum lengths will be at least at least 15 bases, at least 20 bases, at least 25 bases, at least 30 bases, at least 40 bases, at least 50 bases, etc. On the other hand, it should be noted that the actual length may be determined by the analysis engine using the minimum length, the required minimum homology, and the desired minimum melting temperature (for each of the sequences). Depending on the selected length of the consensus sequences, the melting point determination can be carried out using Formula (I) for oligonucleotides with a size of less than 13 bases and according to Formula (II) for oligonucleotides that have a size of equal to or greater than 13 bases.

Tm≈4*(G+C)+2*(A+T)−5 (I)

Tm=64.9+41*(G+C−16.4)/(A+T+G+C) (II)

Primers are typically retained/selected in length such that the oligonucleotides meet or exceed a predetermined melting point (e.g., 65° C.). The so identified consensus sequences are then collected into respective adjusted alignment outputs for the distinct pathogen families Viewed form a different perspective, each pathogen will have its own adjusted alignment output with all of the sequences matching the minimum thresholds for homology, length, and melting temperature. Of course, it should be recognized that where the assay is a multiplex single-pot assay, the predetermined melting temperatures for the first and second distinct pathogen classes are no more than 5° C. apart, more typically no more than 4° C. apart, even more typically no more than 3° C. apart, and most typically no more than 2° C. apart (e.g., same temperature).

The analysis engine in step 140 takes in output files from Clustal X, Clustal W, or Clustal Omega, containing alignments of different pathogen species that typically belong to one pathogen order, family, or genus. Given these alignment files the analysis engine searches for regions of conservation across all members, preferentially identifying regions where the bases are 100% conserved. Failing to find regions that are 100% conserved, the analysis engine will report the highest conserved region. From these regions, the analysis engine will generate potential oligomers of varying sizes: suitable minimum lengths will be at least at least 15 bases, at least 20 bases, at least 25 bases, at least 30 bases, at least 40 bases, at least 50 bases, etc.

In a further step, the so identified and characterized consensus sequences in respective adjusted alignment outputs are then further processed in an analysis engine as shown in step 160 to remove sequences that would match in sequence (or hybridize under PCR conditions) human sequences and/or other sequences that can be reasonably expected to be at least potentially present in a human. Sequence matching can be done in a variety of manners, and all known manners are deemed suitable for use herein. However, particularly suitable matching algorithms include BlastN to identify matching sequences. As will be appreciated, any matching sequences (e.g., sequences with homology of ≥70%, more typically ≥80%, and most typically ≥90%; or Tm difference to human or other virus target of less than 7° C., more typically less than 5° C., and most typically less than 3° C.) are then eliminated from the respective adjusted alignment outputs sequences to so arrive at the respective corresponding pathogen-specific (e.g., virus-specific) alignment outputs. Thus, further processing will help eliminate false positive assay results that may be due to binding of the primers to the host (e.g., human) genome.

Finally, the inventors then perform a set difference analysis on the pathogen-specific alignment outputs 162 and 164 to so obtain respective sets of consensus sequences for the pathogens. Most typically, the set difference analysis 170 is run as set difference and set union operations (typically using FASTA formatted files) that will then produce unique sequences 172 against both pathogen classes suitable for use as primers in diagnostic PCR reactions. It is noted that the term ‘Set’ is a collection of nucleobases representing the sequences found in the previous steps, and the term ‘set difference’ is defined as the elements of one set, B that are not present in another set A. In the present instance, those differences would be oligomers. Set Union is defined as the elements of Set A that are also in Set B. Given these two operations, the Symmetric Set Difference yields all oligomers that do not overlap from Set A and Set B.

Given multiple FASTA formatted files containing oligomers derived from viral sequences, the analysis engine in step 170 will treat each file as a set of oligomers that identify a pathogen family uniquely. Once these sets are created, set difference and set union operations are performed to discover oligomers that belong to multiple sets of pathogen families. These oligomers are then eliminated, as they are unable to uniquely identify one pathogen family from another. The rest of the oligomers are then returned as sets of oligomers that uniquely identify 1 viral family and can then be mixed with other oligomers that uniquely identify a separate pathogen family within the same assay (e.g., a DNA chip).

Of course, it should be appreciated that suitable sequences presented herein may be synthetic oligonucleotides and oligonucleotide analogs, and that all calculations of melting temperatures will consider the changes in temperature due to the different chemistries. For example, the sequences may include a peptide nucleic acid backbone, a sugar-phosphate, or sugar phosphonate/sulfonate backbone. Likewise, the bases in contemplated sequences may be the naturally occurring nucleobases (e.g., adenine, thymine, cytosine, guanine, uracil), but also non-naturally occurring bases making stable or unstable hydrogen bonds with naturally occurring bases (e.g., inosine, iso-C, iso-G, PICS, 3MN, 3FB, MICS, etc.). Furthermore, it should be noted that suitable oligonucleotides may have degenerate bases in one or more position, or have bases that allow for mismatch. Of course, it should be noted that all of the nucleobases will optionally include a radioisotope or other isotope (e.g., NMR-active label). Likewise, while not preferred, it is also contemplated that the backbone may include one or more labeled moieties.

It is still further contemplated that the oligonucleotide will be a single type of oligo, typically a DNA oligomer. However, RNA oligomers or mixed-type oligomers are also considered suitable for use herein. Additionally, it should be noted that suitable oligomers include those that have a affinity marker (e.g., biotin) or other label for direct (e.g., fluorophore, radioisotope) or indirect identification and/or quantification.

In a typical example, where multiplex detection is desired, a kit will include at least one pair of oligonucleotides suitable to produce an amplification or ligation product via a PCR or LCR reaction. As will be readily appreciated, such pairs will be selected to be either specific to a particular strain or serotype of virus, or to cover multiple strains or serotypes. Where multiple pairs of oligonucleotides are used, it is generally contemplated that such pairs will be selected to have minimal or no cross-reactivity between viral targets and/or amplification products, and that such pairs can be used concurrently in a multiplexed reaction. In that regard, multiplexing PCR or LCR will especially be performed using pairs of oligos that will target specific sequences of different viruses (e.g., Ebola virus and InfluenzaA virus).

To cover multiple strains of a virus, it is especially preferred that the oligonucleotides will target highly conserved regions of the viral genome (e.g., RNA-dependent RNA polymerase start structure) and have similar or even identical melting points. Therefore, the inventors contemplate a selection of oligonucleotides that can be employed in a custom assembled kit to readily identify selected viruses. Exemplary sequences and compositions are shown in more detail below. The above examples provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Moreover, as used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Finally, where the specification claims refers to at least one of something selected from the group consisting of A, B, C . . . and N, the text should be interpreted as requiring only one element from the group, not A plus N, or B plus N, etc.

What is claimed is:

1. A method of obtaining sets of primers for differentiation of two unspecified pathogens, each of the unspecified pathogens belonging to distinct virus families, and wherein each virus family comprises multiple distinct virus species and serotypes, comprising:
performing respective multiple sequence alignments for a plurality of genomes of the virus species and serotypes of the respective distinct virus families to produce an alignment output for each of the distinct virus families;
identifying in each alignment output respective consensus sequences having
(i) a homology above a minimum threshold,
(ii) a length above a minimum threshold, and
(iii) a melting temperature above a minimum threshold;
collecting identified consensus sequences into respective adjusted alignment outputs for the distinct virus families;
eliminating from the respective adjusted alignment output sequences with minimum homology to human and human-hosted sequences to so form respective virus-specific alignment outputs;
performing a set difference analysis on the virus-specific alignment outputs to so obtain respective sets of consensus sequences for the unspecified pathogens; and
selecting primer sequences from the respective sets of consensus sequences.

2. The method of claim 1 wherein the multiple sequence alignment is performed using Clustal X, Clustal W, or Clustal Omega.

3. The method of claim 1 wherein the minimum threshold for the homology is at least 97%.

4. The method of claim 1 wherein the minimum threshold for the homology is 100%.

5. The method of claim 1 wherein the minimum threshold for the length is at least 15 bases.

6. The method of claim 1 wherein the minimum threshold for the length is at least 25 bases.

7. The method of claim 1 wherein the minimum threshold for the melting temperature is at least 60° C.

8. The method of claim 1 wherein the minimum threshold for the melting temperature is at least 65° C.

9. The method of claim 1 wherein the minimum threshold for the length is at least 20 bases, the minimum threshold for the homology is at least 97%, and the minimum threshold for the melting temperature is at least 60° C.

10. The method of claim 1 wherein the homology among the consensus sequences is determined by base-wise increments.

11. The method of claim 1 wherein the length of the consensus sequences is variable and is selected above the minimum threshold to achieve the desired melting temperature.

12. The method of claim 1 wherein the step of selecting primer sequences is performed such that amplicons for the distinct families have a length difference of at least 100 bases.

13. The method of claim 1 wherein the step of eliminating is performed using BlastN.

14. The method of claim 1 wherein the minimum homology to human and human-hosted sequences is at least 90%.

15. The method of claim 1 wherein the human-hosted sequence is a viral sequence known or suspected to be present in the human.

16. The method of claim 1 wherein the set difference analysis is performed using Set Difference and Set Union operations.

17. The method of claim 1 wherein the primer sequences are selected to produce an amplicon has a length of between 100 and 800 bases.

18. The method of claim 1 wherein the unspecified viruses belong to two distinct phylogenetic orders.

19. The method of claim 1 further comprising a step of determining a primer sequence to produce a cDNA from a viral RNA.

20. The method of claim 1 wherein the set of primers comprises between one and five primer pairs for each of the unspecified pathogen.
